(19) United States
(12) Patent Application Publication
(10) Pub. No.: US 2025/0022559 A1
(43) Pub. Date: Jan. 16, 2025

(54) METHODS, SYSTEMS, APPARATUSES, AND DEVICES FOR PROVISIONING REPORTS ASSOCIATED WITH MEDICAL PROCEDURES

(71) Applicants: Saul Barry Issenberg, Pembroke Pines, FL (US); Amar Deshpande, Davie, FL (US); Daniel Sussman, Miami, FL (US); Nathaniel Ernstoff, Pompano Beach, FL (US)

(72) Inventors: Saul Barry Issenberg, Pembroke Pines, FL (US); Amar Deshpande, Davie, FL (US); Daniel Sussman, Miami, FL (US); Nathaniel Ernstoff, Pompano Beach, FL (US)

(21) Appl. No.: 18/770,586

(22) Filed: Jul. 11, 2024

Related U.S. Application Data
(60) Provisional application No. 63/512,985, filed on Jul. 11, 2023.

(51) Int. Cl. G16H 15/00; G06F 3/0482; G06F 3/16

(52) U.S. Cl. CPC G16H 15/00; G06F 3/0482; G06F 3/167

(57) ABSTRACT

A system for provisioning reports associated with medical procedures. The system includes device, a processing device, and a storage device. The device may include an output device for presenting a medical data associated with a medical procedure and an input device for detecting an input associated with the medical procedure from a user. The processing device generates a command from the user based on the detecting, analyzes the command, selects a portion of the medical data based on the analyzing of the command, determines an operation required to be performed based on the selecting of the portion of the medical data and the analyzing of the command, performs the operation based on the determining of the operation, and generates a report for the medical procedure using a template based on the performing of the operation. The storage device stores the report.

### FIELD OF THE INVENTION

Generally, the present disclosure relates to the field of data processing. More specifically, the present disclosure relates to methods, systems, apparatuses, and devices for provisioning reports associated with medical procedures.

### BACKGROUND OF THE INVENTION

The field of data processing is technologically important to several industries, business organizations, and/or individuals. In particular, the use of data processing is prevalent for provisioning reports associated with medical procedures.

Existing techniques for provisioning reports associated with medical procedures are deficient with regard to several aspects. Medical procedure time restraints make documentation costly in two aspects: first proceduralist time and second percentage of proceduralist time dedicated to post-operative paperwork. Existing technology is limited with respect to the efficiency of reducing post-operative paperwork.

Many electronic devices have input and output capabilities. These may include a display and keyboard to receive user input and display content, or configured to receive audible voice commands as input from a microphone. However, existing techniques that use one device to supplement the input and output capabilities of another device may be limited with respect to efficiency, usability, or capability for user experiences.

Therefore, there is a need for improved methods, systems, apparatuses, and devices for provisioning reports associated with medical procedures that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a system for provisioning reports associated with medical procedures, in accordance with some embodiments. Accordingly, the system may include at least one device, a processing device, and a storage device. Further, the at least one device may include at least one output device and at least one input device. Further, the at least one output device may be configured for presenting at least one medical data associated with at least one medical procedure. Further, the at least one input device may be configured for detecting at least one input associated with the at least one medical procedure from at least one user. Further, the processing device may be communicatively coupled with the at least one device. Further, the processing device may be configured for generating at least one command from the at least one user based on the detecting. Further, the processing device may be configured for analyzing the at least one command. Further, the processing device may be configured for selecting at least one portion of the at least one medical data based on the analyzing of the at least one command. Further, the processing device may be configured for determining at least one operation required to be performed for the provisioning of the reports for the at least one medical procedure based on the selecting of the at least one portion of the at least one medical data and the analyzing of the at least one command. Further, the processing device may be configured for performing the at least one operation based on the determining of the at least one operation. Further, the processing device may be configured for generating at least one report for the at least one medical procedure using at least one template based on the performing of the at least one operation. Further, the storage device may be communicatively coupled with the processing device. Further, the storage device may be configured for storing the at least one report.

Further disclosed herein is a method for provisioning reports associated with medical procedures, in accordance with some embodiments. Accordingly, the method may include a step of presenting, using at least one output device of at least one device, at least one medical data associated with at least one medical procedure. Further, the method may include a step of detecting, using at least one input device of the at least one device, at least one input associated with the at least one medical procedure from at least one user. Further, the method may include a step of generating, using a processing device, at least one command from the at least one user based on the detecting. Further, the method may include a step of analyzing, using the processing device, the at least one command. Further, the method may include a step of selecting, using the processing device, at least one portion of the at least one medical data based on the analyzing of the at least one command. Further, the method may include a step of determining, using the processing device, at least one operation required to be performed for the provisioning of the reports for the at least one medical procedure based on the selecting of the at least one portion of the at least one medical data and the analyzing of the at least one command. Further, the method may include a step of performing, using the processing device, the at least one operation based on the determining of the at least one operation. Further, the method may include a step of generating, using the processing device, at least one report for the at least one medical procedure using at least one template based on the performing of the at least one operation. Further, the method may include a step of storing, using a storage device, the at least one report.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an platform consistent with various embodiments of the present disclosure.

FIG. 2 is a block diagram of a system 200 for provisioning reports associated with medical procedures, in accordance with some embodiments.

FIG. 3 is a block diagram of the at least one device 202 of the system 200, in accordance with some embodiments.

FIG. 4 is a block diagram of the system 200, in accordance with some embodiments.

FIG. 5 is a block diagram of the at least one input device 304 of the at least one device 202 of the system 200, in accordance with some embodiments.

FIG. 6 is a block diagram of the at least one input device 304 of the at least one device 202 of the system 200, in accordance with some embodiments.

FIG. 7 is a flowchart of a method 700 for provisioning reports associated with medical procedures, in accordance with some embodiments.

FIG. 8 is a flowchart of a method 800 for the provisioning of the reports associated with the medical procedures, in accordance with some embodiments.

FIG. 9 is a flowchart of a method 900 for the provisioning of the reports associated with the medical procedures, in accordance with some embodiments.

FIG. 10 is a flowchart of a method 1000 for the provisioning of the reports associated with the medical procedures, in accordance with some embodiments.

FIG. 11 is a block diagram of a system 1100 for facilitating voice-activated selections of options displayable in heads-up displays, in accordance with some embodiments.

FIG. 12 is a flow diagram of a method 1200 to create new menu options in a head-up display (HUD) and a computer display for the provisioning of the reports associated with the medical procedures, in accordance with some embodiments.

FIG. 13 is a flow diagram of a method 1300 for facilitating speech training for the provisioning of the reports associated with the medical procedures, in accordance with some embodiments.

FIG. 14 is a flow diagram of a method 1400 for generating and displaying a voice activated menu of choices for the provisioning of the reports associated with the medical procedures, in accordance with some embodiments.

FIG. 15 is a user interface 1500 for facilitating voice-activated selections of options displayable in heads-up displays for the provisioning of the reports associated with the medical procedures, in accordance with some embodiments.

FIG. 16 is a user interface 1600 for facilitating voice-activated selections of options displayable in heads-up displays for the provisioning of the reports associated with the medical procedures, in accordance with some embodiments.

FIG. 17 is a user interface 1700 for facilitating voice-activated selections of options displayable in heads-up displays for the provisioning of the reports associated with the medical procedures, in accordance with some embodiments.

FIG. 18 is a user interface 1800 for facilitating voice-activated selections of options displayable in heads-up displays for the provisioning of the reports associated with the medical procedures, in accordance with some embodiments.

FIG. 19 is a flowchart of a method 1900 for facilitating voice-activated selections of options displayable in heads-up displays, in accordance with some embodiments.

FIG. 20 is a block diagram of a computing device for implementing the methods disclosed herein, in accordance with some embodiments.

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of systems, methods, apparatuses, and devices for provisioning reports associated with medical procedures, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor, and at least one actuator. Examples of the one or more client devices and/or the server computer may include, a desktop computer, a laptop computer, a tablet computer, a personal digital assistant, a portable electronic device, a wearable computer, a smartphone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server, etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface, etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, a public database, a private database, and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled, and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal, or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable data (e.g. username, password, passphrase, PIN, secret question, secret answer, etc.) and/or possession of a machine readable secret data (e.g. encryption key, decryption key, bar codes, etc.) and/or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera, and so on. Likewise, the one or more steps may include receiving, using the communication device, the one or more embodied characteristics from one or more biometric sensors.

Further, one or more steps of the method may be automatically initiated, maintained, and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device, etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data, and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview:

The present disclosure describes systems, methods, apparatuses, and devices for provisioning reports associated with medical procedures.

Further, the provisioning of the reports associated with the medical procedures may include voice-activated selection of options displayable in heads-up displays. Further, the disclosed system may be configured to facilitate real-time use of voice commands to select options shown on a virtual monitor in a Heads-Up Display (HUD) to create a report summary. The virtual options are generated by the Scribe Editor system and represent a hierarchical menu logic to create branching type forms leading to only relevant virtual choices being displayed in the virtual HUD. Images taken during the procedure can be inserted into the final report summary-type document stored on a PC/laptop.

Further, the disclosed devices and methods described herein may be related to the selection by voice commands of virtual options (shown as virtual buttons) displayed in an augmented or virtual reality type monitor, and/or a PC/laptop monitor display of information from multiple different devices. A monitor-virtualization system, such as a head-mountable device, (or an ophthalmic device or an intraocular implant,) can render a virtual monitor in augmented, virtual, or mixed reality. The user can apply voice commands to identify and select areas of interest on the input video shown on the main virtual screen. The user can activate a voice-prompted transcription process that uses a hierarchical choice design to select generated options dependent on prior selections to describe an area of interest and the actions taken during a procedure that is stored on files on the system's computer. The transcription system can be applied to post-procedure documentation requirements.

Further, the system may incorporate a voice recognition training system that is self-learning. It is based on accepting unknown words that are used during a medical procedure, and identifying when they are an alias for a recognized term. Such aliases are then stored in a user database so that they can be immediately acted on if they are detected in the future.

Further, the real-time use of voice commands to select options shown on a monitor to create a report summary. The options are generated by the Script Editor system and represent a hierarchical menu logic to create branching type forms leading to only relevant virtual choices being displayed on the monitor. Images/diagrams/graphics/tables etc. can be inserted into the final report summary type document stored on a PC/laptop.

The present disclosure describes a technique related to medical devices and systems attached to a head-mounted display that uses a voice command to select an area from a chosen virtual monitor displaying data in the HUD from one of the medical devices and then activate using a voice command a transcribe system in general and to prompt the user to voice select generated menu options that appear in the HUD based on prior selections, in particular. The virtual menu is a hierarchical choice system created on a PC Scribe Editor and is customizable to reflect the logical flow of medical discovery and required documentation for a procedure. Key inputs on the Script Editor will generate virtual menu options in the HUD. New menu options are generated depending on prior selection by the user. The Scribe Editor allows the user to create a procedure template with key inputs for the virtual menu options and text boxes to embed the option choices in sentences when a particular option is selected during the procedure. The generated transcription report from the procedure includes the choices made and allows for images (snapshots) taken of the area of interest selected to be embedded in the body of the generated report at the relevant place.

Further, the disclosed system uses a first device head-mounted display (HUD) to augment the physical monitors of multiple devices simultaneously in a computer-generated reality (CGR). In some implementations, areas of interest can be selected on the virtual monitor and a transcript added using voice activation commands by the user that describes the anatomic condition found and actions taken by the user. HUDs are wearable electronic displays that project an image on the wearer's field of view. In virtual reality applications, the projected image typically blocks the field of view of the wearer. In augmented reality applications, a projected image is typically superimposed on a portion of the wearer's field of view, allowing the wearer to simultaneously view real and virtual features. In video see-through or video pass-through devices the wearer's view is a video of the real world with an augmented projected image superimposed on a portion of the video, allowing the wearer to simultaneously view video of real and virtual features.

Further, the disclosed system may use a microphone input to capture the user's voice commands and then output these voice commands as actions to select augmented menu options that can for example activate a transcription to a segment of a virtual input. Further, in some embodiments, comprise user interfaces, such as a graphical user interface (GUI) and/or voice-activated user interface enabled and trained by the voice-activation software engine. This software is part of the system. Interaction with a GUI is through voice and/or virtual hand.

Further, in some embodiments, an electronic device uses a processor to perform a method of using a first device to provide a CGR environment that augments the input and output capabilities of other devices. The method involves obtaining images of a physical environment (e.g., video for video passthrough) and identifying a device in the physical environment. For example, the second device may be detected via proximity-based detection or video input. The method presents a CGR environment that includes a view of at least a portion of the images and that augments an output capability or an input capability of another device. In some examples, the HUD device can record the user selections through a transcription option allowing a report to be generated at the end of a session.

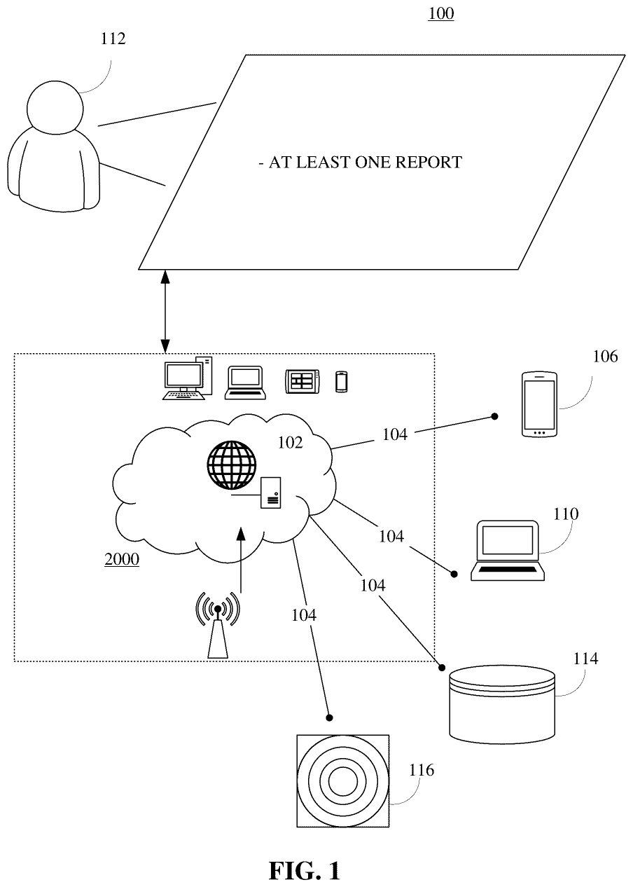

FIG. 1 is an illustration of an platform 100 consistent with various embodiments of the present disclosure. By way of non-limiting example, the platform 100 to facilitate provisioning reports associated with medical procedures may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, and sensors 116 over a communication network 104, such as, but not limited to, the Internet. Further, users of the platform 100 may include relevant parties such as, but not limited to, end-users, administrators, service providers, service consumers, users, doctors, surgeons, patients, and so on. Accordingly, in some instances, electronic devices operated by the one or more relevant parties may be in communication with the platform.

A user 112, such as the one or more relevant parties, may access platform 100 through a software application or browser. The software application may be embodied as, for example, but not be limited to, a website, a web application, a desktop application, and a mobile application compatible with a computing device 2000.

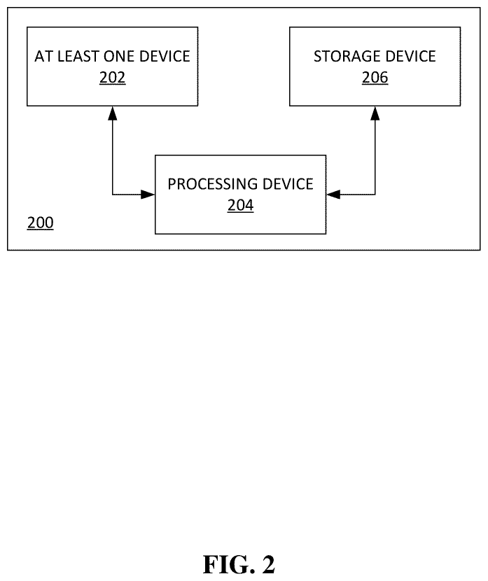

FIG. 2 is a block diagram of a system 200 for provisioning reports associated with medical procedures, in accordance with some embodiments. Accordingly, the system 200 may include at least one device 202, a processing device 204, and a storage device 206.

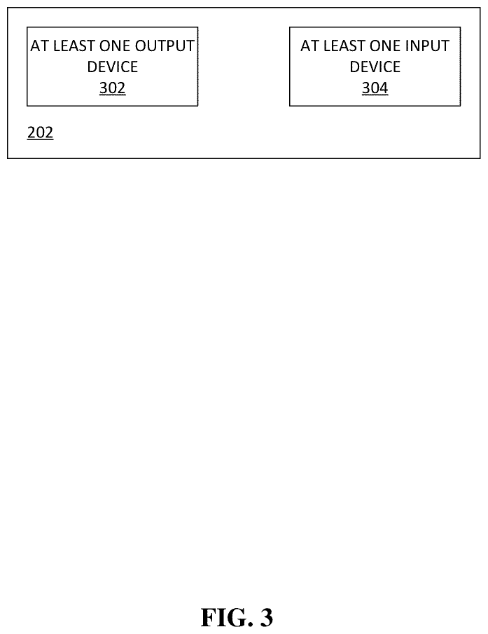

Further, the at least one device 202 may include at least one output device 302 and at least one input device 304, as shown in FIG. 3. Further, the at least one output device 302 may be configured for presenting at least one medical data associated with at least one medical procedure. Further, the at least one input device 304 may be configured for detecting at least one input associated with the at least one medical procedure from at least one user. Further, the at least one device 202 may include a wearable device, an extended reality device, etc. Further, the at least one output device 302 may include a display device, a head mounted display (HUD), etc. Further, the at least one output device 302 may include a screen (such as a virtual screen, an extended reality screen, a mixed reality screen, a display screen (a monitor screen), etc.). Further, the at least one output device 302 may include a heads up display. Further, the at least one medical procedure may include a procedure, a real time procedure, etc. Further, the at least one user may include a surgeon, a doctor, a wearer, etc. Further, the at least one user may include an individual. Further, the at least one medical data may include at least one video (such as an input video, a video input, etc.) associated with the at least one medical procedure. Further, the presenting of the at least one medical data may include streaming of the at least one video in real time.

Further, the processing device 204 may be communicatively coupled with the at least one device 202. Further, the processing device 204 may be configured for generating at least one command from the at least one user based on the detecting. Further, the at least one command may include a voice command input. Further, the processing device 204 may be configured for analyzing the at least one command. Further, the processing device 204 may be configured for selecting at least one portion of the at least one medical data based on the analyzing of the at least one command. Further, the at least one portion of the at least one medical data may include an area of interest in the at least one video. Further, the at least one portion of the at least one medical data may include at least one image of the at least one video, at least one portion of the at least one image of the at least one video, at least one section of the at least one image of the at least one video, etc. Further, the processing device 204 may be configured for determining at least one operation required to be performed for the provisioning of the reports for the at least one medical procedure based on the selecting of the at least one portion of the at least one medical data and the analyzing of the at least one command. Further, the at least one operation may include transcribing the at least one medical procedure, describing the at least one portion of the at least one medical data, etc. Further, the processing device 204 may be configured for performing the at least one operation based on the determining of the at least one operation. Further, the processing device 204 may be configured for generating at least one report for the at least one medical procedure using at least one template based on the performing of the at least one operation. Further, the at least one report may include a report summary of the at least one medical procedure. Further, the at least one report may include a transcription, a description, etc., associated with the at least one medical procedure, the at least one portion of the at least one medical data, etc., the at least one portion of the at least one medical data, etc.

Further, the storage device 206 may be communicatively coupled with the processing device 204. Further, the storage device 206 may be configured for storing the at least one report.

Further, in some embodiments, the processing device 204 may be configured for generating a menu structure comprising at least one hierarchy level. Further, each of the at least one hierarchy level may include a plurality of menu options corresponding to each of the at least one hierarchy level. Further, the processing device 204 may be configured for selecting a menu option from the plurality of menu options for each of the at least one hierarchy level based on the at least one command. Further, the determining of the at least one operation may be further based on the selecting of the menu option for each of the at least one hierarchy level. Further, the at least one output device 302 may be further configured for presenting the menu structure based on the generating of the menu structure. Further, the menu structure may include a hierarchical menu comprising a hierarchical menu logic. Further, the menu structure may include a virtual menu. Further, the plurality of menu options for each of the at least one hierarchy level may be associated with a procedure, a finding, a location, a description, etc. Further, the plurality of menu options for each of the at least one hierarchy level describes one or more anatomic conditions found during the at least one medical procedure and one or more actions taken by the at least one user during the at least one medical procedure. Further, in some embodiments, the processing device 204 may be configured for generating a user navigation path based on the selecting of the menu option from the plurality of menu options for each of the at least one hierarchy level. Further, the determining of the at least one operation may be further based on the user navigation path.

Further, in an embodiment, the processing device 204 may be further configured for analyzing the at least one medical data. Further, the generating of the menu structure may be further based on the analyzing of the at least one medical data.

Further, in an embodiment, the at least one hierarchy level may include a first hierarchy level. Further, the plurality of menu options may include a plurality of first menu options for the first hierarchy level. Further, the presenting of the menu structure further may include presenting the menu structure with the first hierarchy level. Further, the selecting of the menu option from the plurality menu options for each of the at least one hierarchy level may include selecting a first menu option from the plurality of menu options for the first hierarchy level based on a first command of the at least one command. Further, the selecting of the menu option from the plurality menu options for each of the at least one hierarchy level may include selecting a subsequent menu option from a plurality of subsequent menu options for each of at least one subsequent hierarchy level of the at least one hierarchy level based on at least one subsequent command of the at least one command. Further, the generating of the menu structure may include generating the at least one subsequent hierarchy level after the first hierarchy level based on the selecting of the first menu option from the plurality of first menu options for the first hierarchy level. Further, each of the at least one subsequent hierarchy level may include the plurality of subsequent menu options for each of the at least one subsequent hierarchy level. Further, the presenting of the menu structure may include presenting the menu structure with the at least one subsequent hierarchy level based on the generating of the at least one subsequent hierarchy level.

Further, in an embodiment, the at least one operation may include a generation of the at least one description for the at least one portion of the at least one medical data based on the menu option selected from the plurality of menu options for each of the at least one hierarchy level. Further, the at least one description corresponds to the menu option selected for each of the at least one hierarchy level.

Further, in an embodiment, the processing device 204 may be configured for determining at least one template element based on the selecting of the menu option for each of the at least one hierarchy level. Further, the at least one template element corresponds to the menu option for each of the at least one hierarchy level. Further, the processing device 204 may be configured for generating the at least one template based on the determining of the at least one template. Further, the generating of the at least one report may be further based on the generating of the at least one template. Further, the at least one template may include a word template.

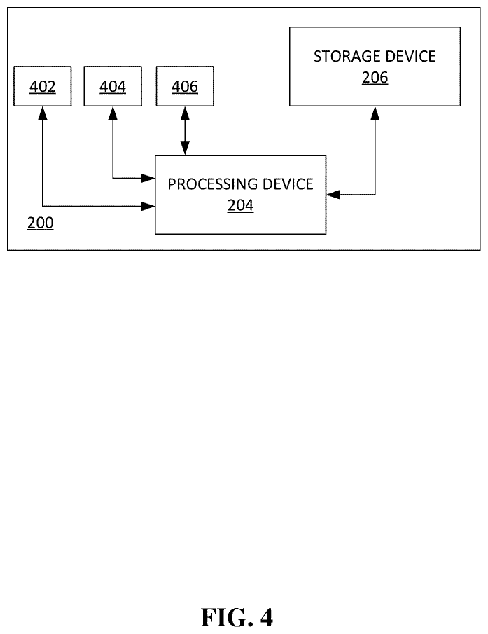

Further, in some embodiments, the at least one device 202 may include at least one of a head mountable device 402, an ophthalmic device 404, and an implantable device 406, as shown in FIG. 4. Further, the head mountable device 402 may include a head-mounted display device. Further, the implantable device 406 may include an intraocular implant.

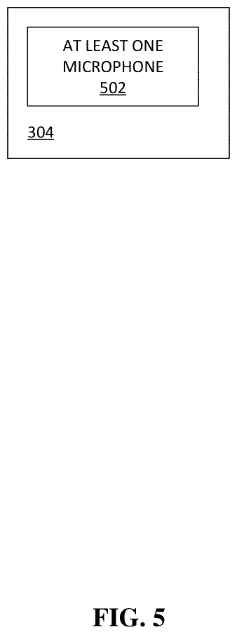

Further, in some embodiments, the at least one input device 304 may include at least one microphone 502, as shown in FIG. 5. Further, the at least one input may include at least one utterance of the at least one user.

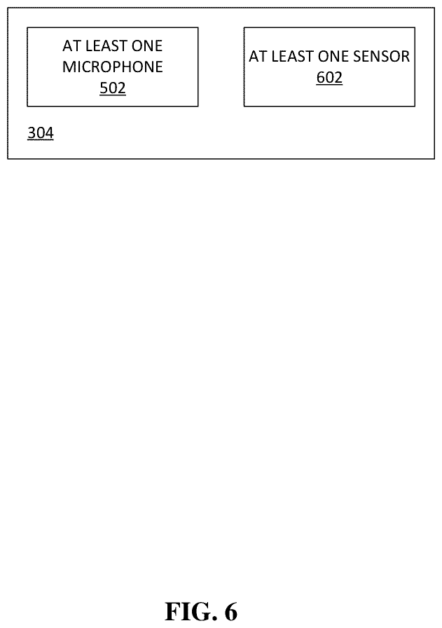

Further, in an embodiment, the at least one input device 304 may include at least one sensor 602, as shown in FIG. 6. Further, the at least one sensor 602 may be configured for detecting at least one action performed by the at least one user during the at least one medical procedure. Further, the at least one sensor 602 may include an image sensor (camera), a gesture sensor, a motion sensor, a movement sensor, a gaze tracking sensor, etc. Further, the at least one action may include a hand gesture, a hand movement, a facial expression, a gaze, etc. Further, the processing device 204 may be configured for generating at least one action data for the at least one action based on the detecting of the at least one action. Further, the processing device 204 may be configured for analyzing the at least one action data. Further, the performing of the at least one operation may be further based on the analyzing of the at least one action data.

Further, in some embodiments, the processing device 204 may be further configured for generating at least one content based on the at least one portion of the at least one medical data and the at least one command. Further, the generating of the at least one report may be further based on the at least one content. Further, the at least one content may include text, image, table, etc.

FIG. 3 is a block diagram of the at least one device 202 of the system 200, in accordance with some embodiments.

FIG. 4 is a block diagram of the system 200, in accordance with some embodiments.

FIG. 5 is a block diagram of the at least one input device 304 of the at least one device 202 of the system 200, in accordance with some embodiments.

FIG. 6 is a block diagram of the at least one input device 304 of the at least one device 202 of the system 200, in accordance with some embodiments.

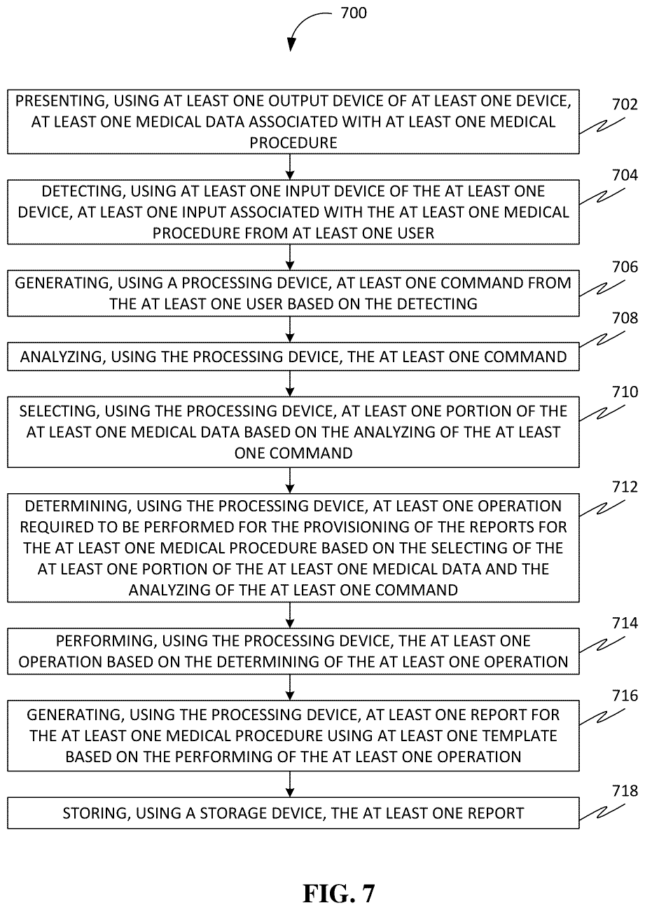

FIG. 7 is a flowchart of a method 700 for provisioning reports associated with medical procedures, in accordance with some embodiments. Accordingly, at 702, the method 700 may include presenting, using at least one output device of at least one device, at least one medical data associated with at least one medical procedure.

Further, at 704, the method 700 may include detecting, using at least one input device of the at least one device, at least one input associated with the at least one medical procedure from at least one user.

Further, at 706, the method 700 may include generating, using a processing device, at least one command from the at least one user based on the detecting.

Further, at 708, the method 700 may include analyzing, using the processing device, the at least one command.

Further, at 710, the method 700 may include selecting, using the processing device, at least one portion of the at least one medical data based on the analyzing of the at least one command.

Further, at 712, the method 700 may include determining, using the processing device, at least one operation required to be performed for the provisioning of the reports for the at least one medical procedure based on the selecting of the at least one portion of the at least one medical data and the analyzing of the at least one command.

Further, at 714, the method 700 may include performing, using the processing device, the at least one operation based on the determining of the at least one operation.

Further, at 716, the method 700 may include generating, using the processing device, at least one report for the at least one medical procedure using at least one template based on the performing of the at least one operation.

Further, at 718, the method 700 may include storing, using a storage device, the at least one report.

Further, in some embodiments, the at least one device may include at least one of a head mountable device, an ophthalmic device, and an implantable device.

Further, in some embodiments, the at least one input device may include at least one microphone. Further, the at least one input may include at least one utterance of the at least one user.

In further embodiments, the method 700 may include generating, using the processing device, at least one content based on the at least one portion of the at least one medical data and the at least one command. Further, the generating of the at least one report may be further based on the at least one content.

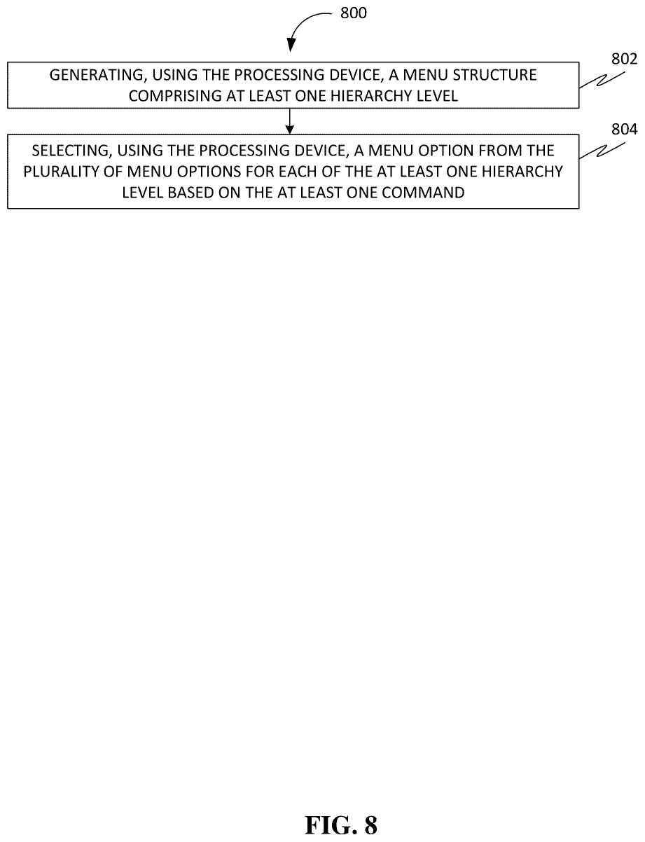

FIG. 8 is a flowchart of a method 800 for the provisioning of the reports associated with the medical procedures, in accordance with some embodiments. Accordingly, at 802, the method 800 may include generating, using the processing device, a menu structure comprising at least one hierarchy level. Further, each of the at least one hierarchy level may include a plurality of menu options corresponding to each of the at least one hierarchy level.

Further, at 804, the method 800 may include selecting, using the processing device, a menu option from the plurality of menu options for each of the at least one hierarchy level based on the at least one command. Further, the determining of the at least one operation may be further based on the selecting of the menu option for each of the at least one hierarchy level. Further, the at least one output device may be further configured for presenting the menu structure based on the generating of the menu structure.

In further embodiments, the method 800 may include analyzing, using the processing device, the at least one medical data. Further, the generating of the menu structure may be further based on the analyzing of the at least one medical data.

Further, in some embodiments, the at least one hierarchy level may include a first hierarchy level. Further, the plurality of menu options may include a plurality of first menu options for the first hierarchy level. Further, the presenting of the menu structure further may include presenting the menu structure with the first hierarchy level. Further, the selecting of the menu option from the plurality menu options for each of the at least one hierarchy level may include selecting a first menu option from the plurality of menu options for the first hierarchy level based on a first command of the at least one command. Further, the selecting of the menu option from the plurality menu options for each of the at least one hierarchy level may include selecting a subsequent menu option from a plurality of subsequent menu options for each of at least one subsequent hierarchy level of the at least one hierarchy level based on at least one subsequent command of the at least one command. Further, the generating of the menu structure may include generating the at least one subsequent hierarchy level after the first hierarchy level based on the selecting of the first menu option from the plurality of first menu options for the first hierarchy level. Further, each of the at least one subsequent hierarchy level may include the plurality of subsequent menu options for each of the at least one subsequent hierarchy level. Further, the presenting of the menu structure further may include presenting the menu structure with the at least one subsequent hierarchy level based on the generating of the at least one subsequent hierarchy level.

Further, in some embodiments, the at least one operation may include a generation of the at least one description for the at least one portion of the at least one medical data based on the menu option selected from the plurality of menu options for each of the at least one hierarchy level. Further, the at least one description corresponds to the menu option selected for each of the at least one hierarchy level.

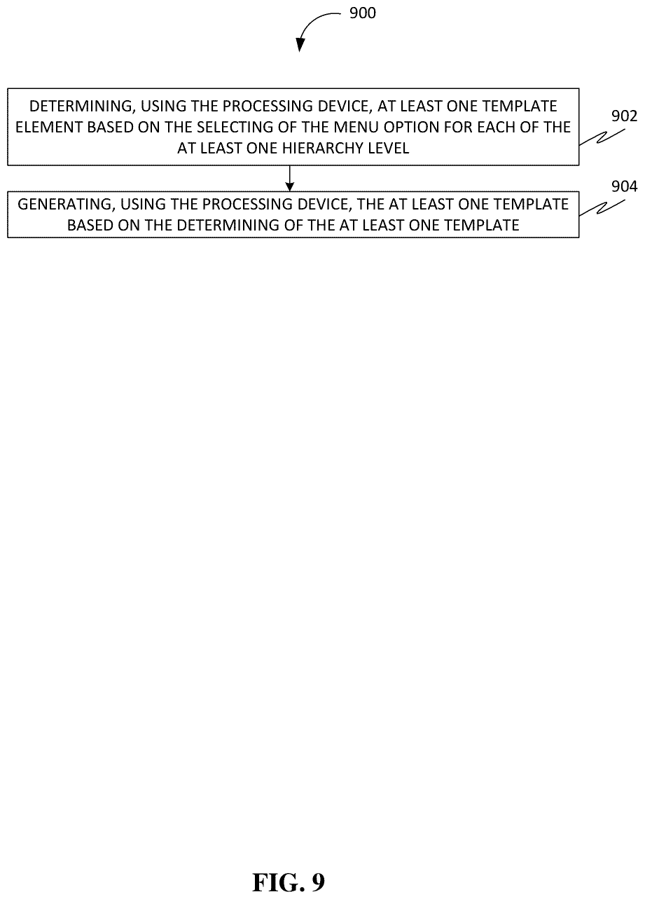

FIG. 9 is a flowchart of a method 900 for the provisioning of the reports associated with the medical procedures, in accordance with some embodiments. Accordingly, at 902, the method 900 may include determining, using the processing device, at least one template element based on the selecting of the menu option for each of the at least one hierarchy level. Further, the at least one template element corresponds to the menu option for each of the at least one hierarchy level.

Further, at 904, the method 900 may include generating, using the processing device, the at least one template based on the determining of the at least one template. Further, the generating of the at least one report may be further based on the generating of the at least one template.

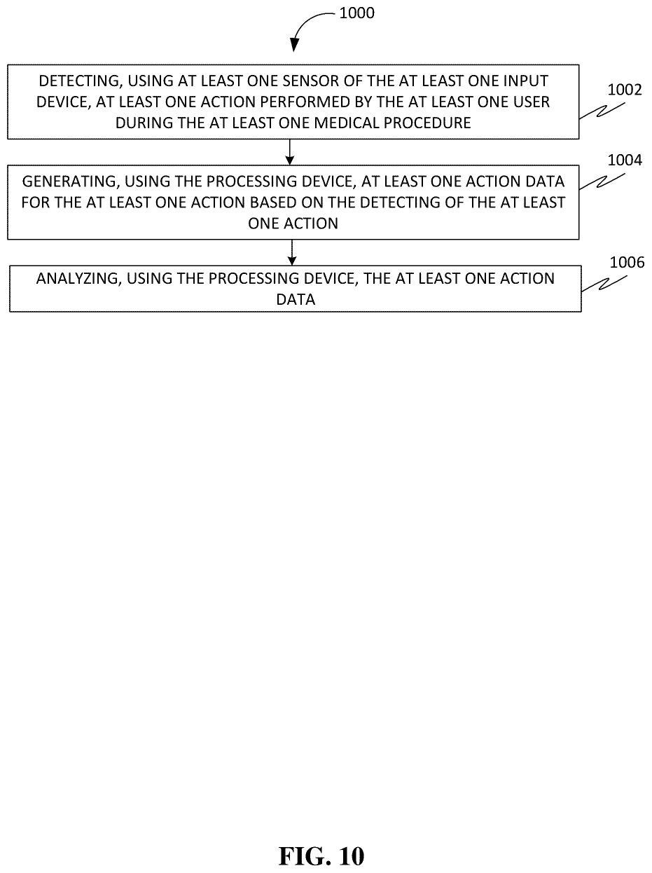

FIG. 10 is a flowchart of a method 1000 for the provisioning of the reports associated with the medical procedures, in accordance with some embodiments. Accordingly, at 1002, the method 1000 may include detecting, using at least one sensor of the at least one input device, at least one action performed by the at least one user during the at least one medical procedure.

Further, at 1004, the method 1000 may include generating, using the processing device, at least one action data for the at least one action based on the detecting of the at least one action.

Further, at 1006, the method 1000 may include analyzing, using the processing device, the at least one action data. Further, the performing of the at least one operation may be further based on the analyzing of the at least one action data.

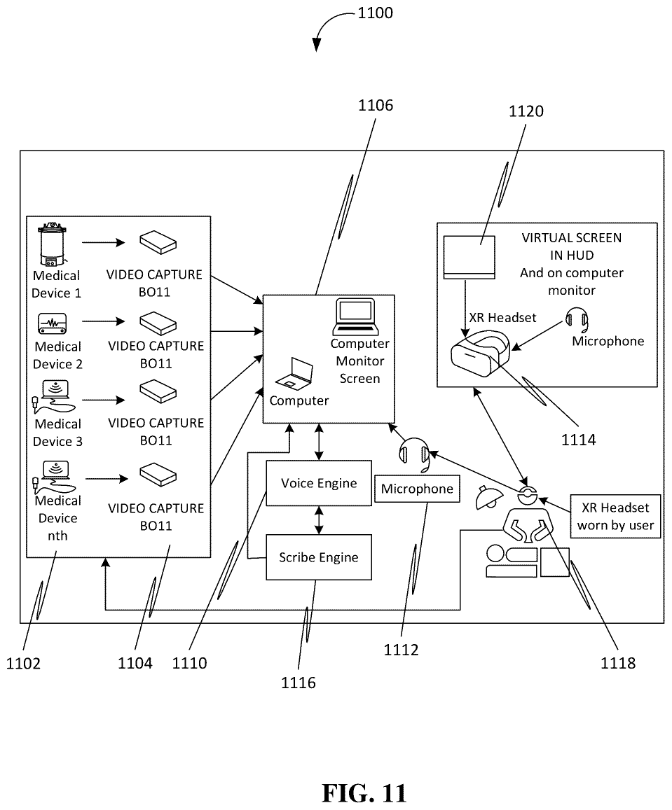

FIG. 11 is a block diagram of a system 1100 for facilitating voice-activated selections of options displayable in heads-up displays, in accordance with some embodiments. Accordingly, the system 1100 may include at least one medical device 1102 comprising a N number of medical devices (or data output sources). Further, the system 1100 may include at least one video capture device 1104 comprising a N number of video capture devices. Further, the system 1100 may include at least one computer 1106. Further, the at least one computer 1106 may include a computer monitor screen. Further, the system 1100 may include at least one head-mounted display device 1114 such as an extended reality (XR) head-mounted display. Further, the at least one head-mounted display device 1114 may include a virtual screen. Further, the at least one computer 1106 may be coupled to the at least one head-mounted display device 1114 via at least one microphone 1112. Further, the at least one head-mounted display device 1114 may be configured to be worn by at least one user 1118 such as a user. Further, the system 1100 may include at least one voice engine 1110 such as a voice engine UI with at least one training component installed on the at least one computer 1106 configured to respond to and record a plurality of voice commands of the at least one user 1118. Further, the plurality of voice commands may be received by the at least one computer 1106 by inputting a plurality of choices into a scribe engine 1116, such as a scribe engine, that creates a summary document stored on the at least one computer 1106 and generates a plurality of new options for the at least one user 1118 to select in at least one head-mounted display device 1114. Further, the system 1100 may include the at least one microphone 1112 to pick up the plurality of voice commands from the at least one user 1118. Further, the system 1100 may include at least one user interface 1120. Further, the at least one user interface 1120 may be comprised in the at least one head-mounted display device 1114 and a plurality of multiple video inputs. Further, the at least one user interface 1120 may be configured to provide to the at least one computer 1106 at least one voice input received from the at least one head-mounted display device 1114 in association with at least one virtual hand motion input received from at least one hand tracking device, to perform in system mode an action of relaying to the at least one computer 1106. Further, a plurality of actions initiated by the plurality of voice commands to transcribe a plurality of findings of at least one surgeon through a series of logic UI voice choices presenting in a real-time procedure report for the at least one user 1118.

Further, in some embodiments, the at least one head-mounted display device 1114 may include at least one at least one head-up display (HUD).

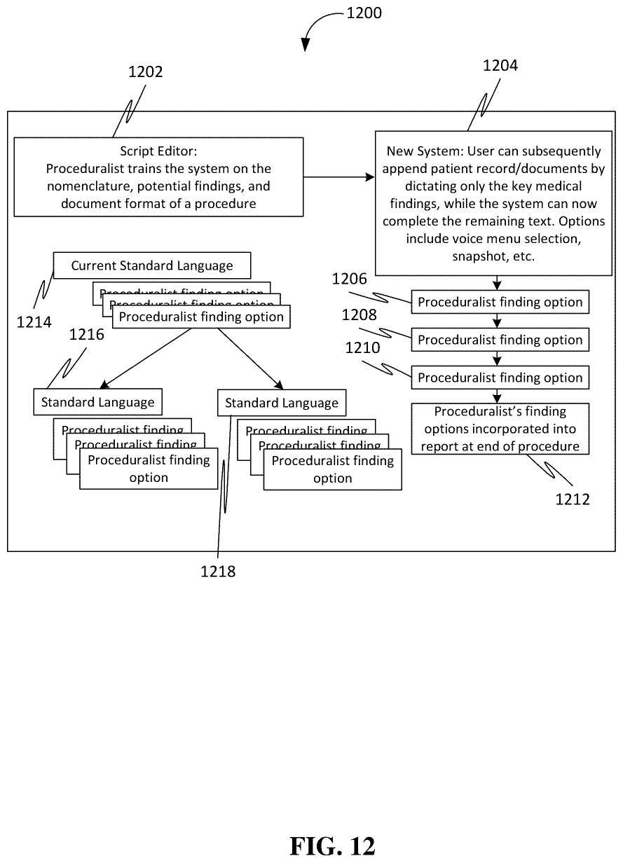

FIG. 12 is a flow diagram of a method 1200 to create new menu options in a head-up display (HUD) and a computer display for the provisioning of the reports associated with the medical procedures, in accordance with some embodiments.

Further, at 1202, the method 1200 may include proceduralist training the system on the nomenclature, potential findings, and document format of a procedure. Further, at 1204, the method 1200 may include a user subsequently appending the patient's record/documents by dictating only the key medical findings, while the system can now complete the remaining text. Options include voice menu selection, snapshot, etc. Further, at 1206, the method 1200 may include proceduralist finding option. Further, at 1208, the method 1200 may include proceduralist finding option. Further, at 1210, the method 1200 may include proceduralist finding option. Further, at 1212, the method 1200 may include proceduralist's finding options incorporated into a report at the end of a procedure. Further, the method 1200 may include a plurality of proceduralist finding options associated with a current standard language 1214. Further, for the current standard language 1214, there are two standard languages 1216-1218. Further, each of the two standard languages 1216-1218 may be associated with a plurality of proceduralist finding options.

Further, in some embodiments, the method 1200 may be associated with a script engine (such as a script engine system) for facilitating voice-activated selections of options displayable in heads-up displays. Further, the script engine (or script editor) may be connected to a voice engine system to perform a method (such as the method 1200) integrated with a voice training system to create a plurality of new menu options in a HUD and computer display. Further, the script. Further, the voice engine system may allow a user to create a custom path of menu choices to interactively navigate the user through a path of menu choices using a plurality of voice commands on a user interface to lead the user through a computer application. Further, the user interface may include at least one of a virtual user interface and a real user interface. Further, the method may be performed automatically using at least one hardware processor. Further, in some embodiments, the method may include displaying a current menu of choices in a first area of a user interface display.

Further, the method may include a selection of a first menu item choice that may auto-populate a second menu selection option based on the first menu item choice and displayed in the first area of the user interface display.

Further, the method may create the custom path of menu choices and text to interactively navigate the user through the path of menu choices using the plurality of voice commands on the user interface to lead the user through the computer application. Further, the method may be performed automatically by the at least one hardware processor. Further, in some embodiments, the method may include displaying the current menu of choices in the first area of the user interface display.

Further, the method may include displaying on a second area of the user interface display, a flow chart diagram of past inputs for a plurality of user-created choices or text represented in box form. Further, the method may include allowing the user to connect these said boxes in a user-determined logic form. In an embodiment, the first area and the second area may be viewable concurrently on the user interface display.

Furthermore, the method may create a word template that may reflect a specific user navigation path of menu choices and custom text created by a user creator. Further, a user creator may be an individual using the method within to create the menu choices, the texts, and the word templates.

Further, the method may include allowing a user to select a menu text item from the current menu of choices displayed on the first area of the user interface display to add a text to create a text box on the flow chart in the second area.

Further, the method may include allowing a user to select a menu option item from the current menu of choices displayed on the first area of the user interface display and to drill down through levels of menu choices based on the selected menu item from a prior list of menu choices. Further, the first area menu selection choice is bifurcated into a sub-first area and a sub-second area that may be scrollable. Further, in the sub-first area the user creates titles for choices, and in the sub-second area the user creates choices. Further, the selections may be displayed in an option box on the flow chart in the second area of the user interface display.

Further, the method may include storing the created flowchart of option choices in a file that may be stored on a computer. Further, the user may select the file by a voice command to the virtual menu shown in the HUD or on a computer monitor, the first menu of option choices may be displayed as text inside a box in the virtual screen or computer monitor. Further, a first choice item may be selected, a second menu selection option based on the first menu item choice and displayed in the first area of a user interface display replacing the first choice item list. This process continues until no further choice items may be available.

Further, each menu option includes a “RETRY” option allowing the user to return to the previous list of menu options.

Furthermore, text that may be stored on the computer representing the user's choice navigation may be displayed in a separate window on the HUD display or in the computer monitor user interface display.

Further, on reaching the end of the menu choices, the user may receive a request to confirm selection choices as correct. Furthermore, once selected the user's choice selection may be stored on the computer where the selections populate the initial selected scribe template file on a document stored against that user.

Furthermore, a snapshot taken immediately before or after the scribe option is selected is inserted below the text in the document stored against the user.

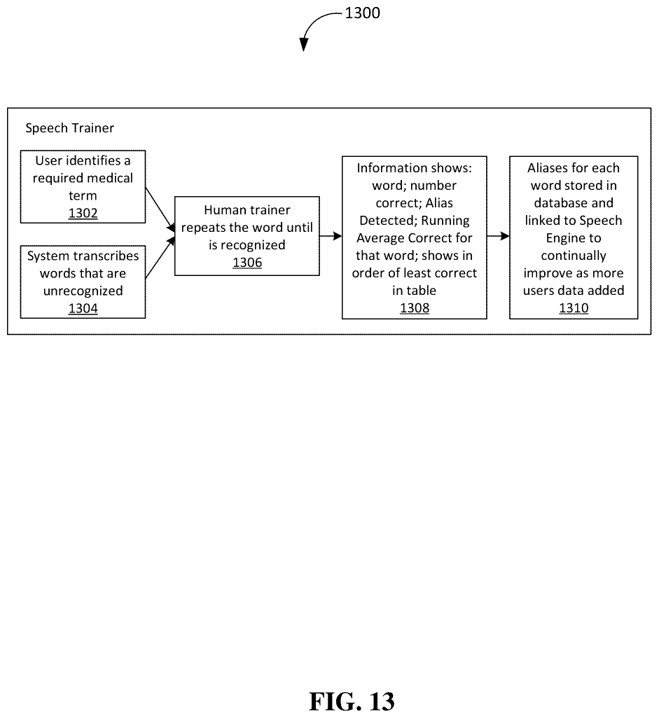

FIG. 13 is a flow diagram of a method 1300 for facilitating speech training for the provisioning of the reports associated with the medical procedures, in accordance with some embodiments. Further, a user identifies a required medical term in step 1302 of the method 1300. Further, a system transcribes words that are unrecognized in step 1304 of the method 1300. Further, a human trainer repeats the word until is recognized in step 1306 of the method 1300. Further, the step 1306 is performed after the steps 1302 and 1304. Further, information is shown such as word; number correct; Alias Detected; Running Average Correct for that word in step 1308 of the method 1300. Further, the information is shown in order of least corrent in a table. Further, aliases for each word are stored in a database and linked to Speech Engine to continually improve as more user data is added in step 1310 of the method 1300.

Further, in some embodiments, the method 1300 may be associated with a speech training system for facilitating voice-activated selections of options displayable in heads-up displays. Further, the speech training system may be configured for automatically performing a method (such as the method 1300) by at least one hardware processor. Further, the method may include capturing a plurality of input option words created by a user and displayed in a plurality of option boxes on the flow chart of the voice engine system and adding them to the speech training system.

Further, in some embodiments, the speech training system may be configured for allowing the user to select from a menu item to add a new word to the speech trainer that may be displayed on the first area of the user interface display.

Further, in some embodiments, the speech training system matches input words to a library of words or suggests aliases for the word. Further, the word library and training results may be stored on a memory device operatively coupled to the hardware processor. Further, the speech training system may be configured for displaying a current menu of aliases on a first portion of a user interface display and showing the running total of correct and alias attempts for each word.

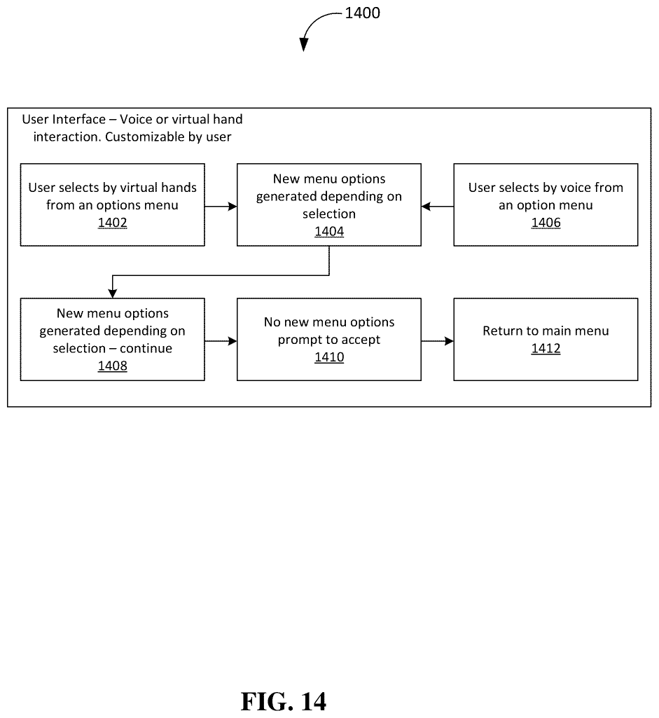

FIG. 14 is a flow diagram of a method 1400 for generating and displaying a voice activated menu of choices for the provisioning of the reports associated with the medical procedures, in accordance with some embodiments. Further, a user selects by virtual hands from an options menu in step 1402 of the method 1400. Further, new menu options generated depending on the selection in step 1404 of the method 1400. Further, the generation of the new menu options is continued depending on the selection in step 1408 of the method 1400 after the step 1404. Further, the user selects by voice from an option menu after the new menu options are generated in step 1406 of the method 1400. Further, no new menu options prompt to accept in step 1410 of the method 1400. Further, return to main menu in step 1412 of the method 1400.

Further, in some embodiments, the method 1400 may be associated with a virtual interface. Further, the virtual interface may be associated with a virtual interface system. Further, the virtual interface system facilitates voice-activated selections of options displayable in heads-up displays. Further, the virtual interface system may be configured for generating in a HUD (and/or on a computer monitor screen) a voice-activated menu of choices and to display the current menu of choices in the first area of the user's virtual HUD (and the computer monitor screen.). Further, the virtual interface system may be configured for automatically performing a method (such as the method 1400) by at least one hardware processor to allow the user to voice select a menu item from a current menu of choices displayed in the first area of the UI virtual display and (or in the computer monitor screen UI) and drill down through the menu level choices based on a selection of a menu choice from a prior level choice.

Further, the virtual interface system may include a microphone configured to detect a voice command comprised in an input.

Further, in some embodiments, the computer may be configured to display a menu overlaid on an image on the head-mounted display (and the computer monitor screen.)

Further, in some embodiments, the virtual interface system allows the surgeon to select by voice a transcribe option. Further, the virtual interface system uses hierarchical menu logic to create branching type forms leading to only relevant virtual choices being displayed in the virtual HUD.

Further, in some embodiments, the virtual interface system allows the user to customize the logic to create new type forms.

Further, in some embodiments, the system allows the user to customize the template for the transcribe option with customized inputs and type forms.

Further, in many embodiments, the techniques described herein may provide a practical application and several technological improvements. In one example, the techniques described herein may provide for improved efficiency for the user, by providing a method of recording voice notes during a procedure that may be computer transcribed into a report post-procedure.

Further, in one embodiment, the techniques described herein may facilitate increased safety by use of a CGR environment, by providing a real-time notation of medical conditions and procedures.

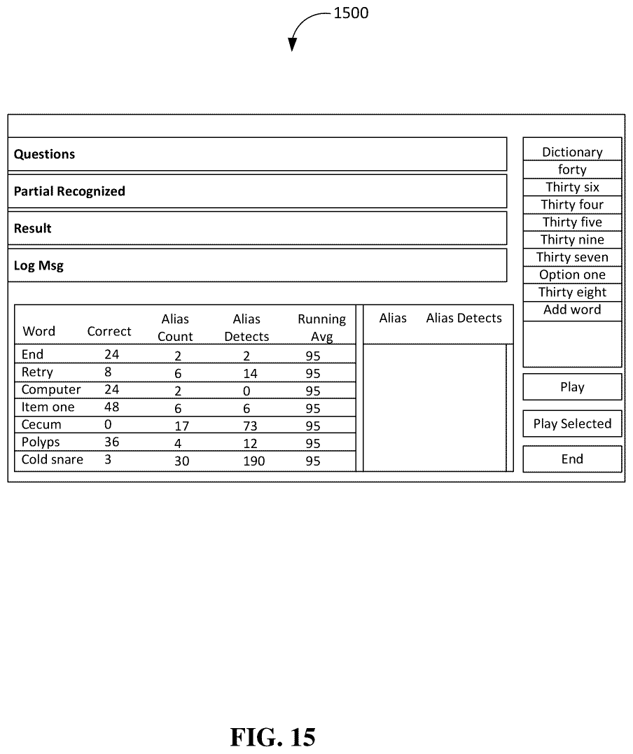

FIG. 15 is a user interface 1500 for facilitating voice-activated selections of options displayable in heads-up displays for the provisioning of the reports associated with the medical procedures, in accordance with some embodiments.

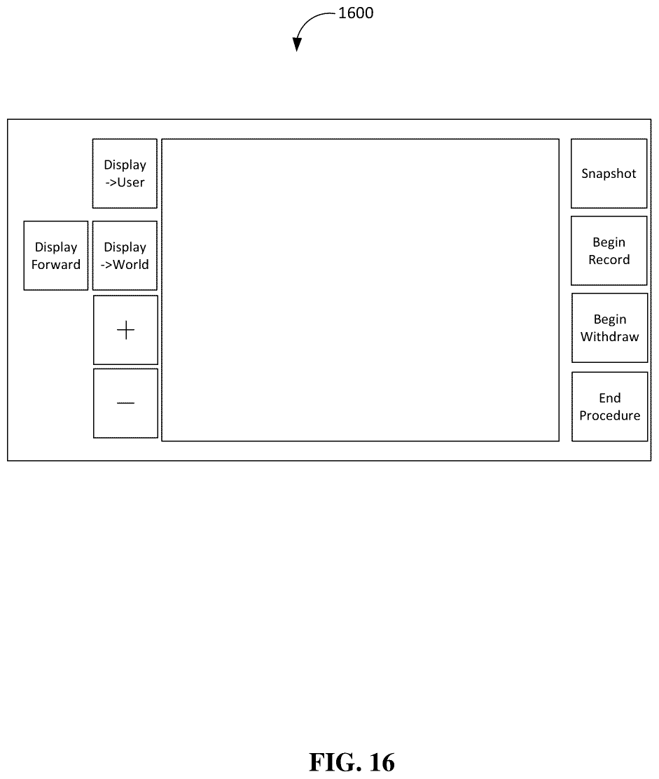

FIG. 16 is a user interface 1600 for facilitating voice-activated selections of options displayable in heads-up displays for the provisioning of the reports associated with the medical procedures, in accordance with some embodiments.

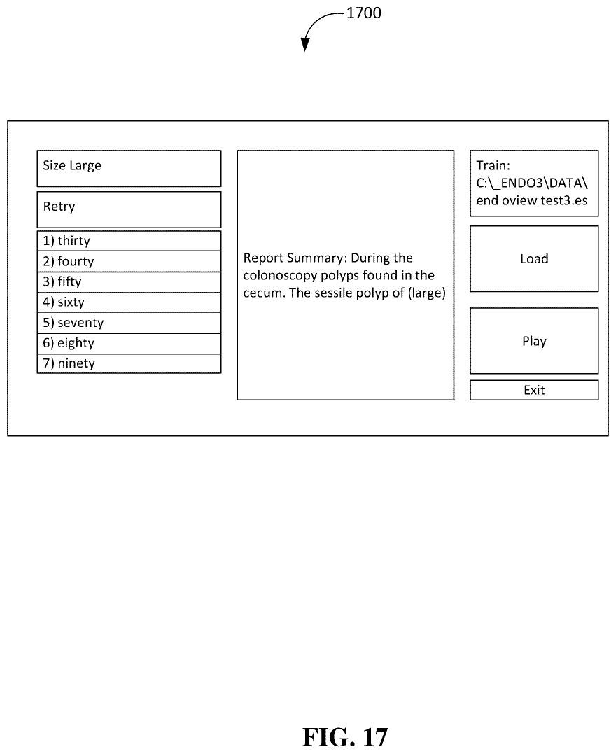

FIG. 17 is a user interface 1700 for facilitating voice-activated selections of options displayable in heads-up displays for the provisioning of the reports associated with the medical procedures, in accordance with some embodiments.

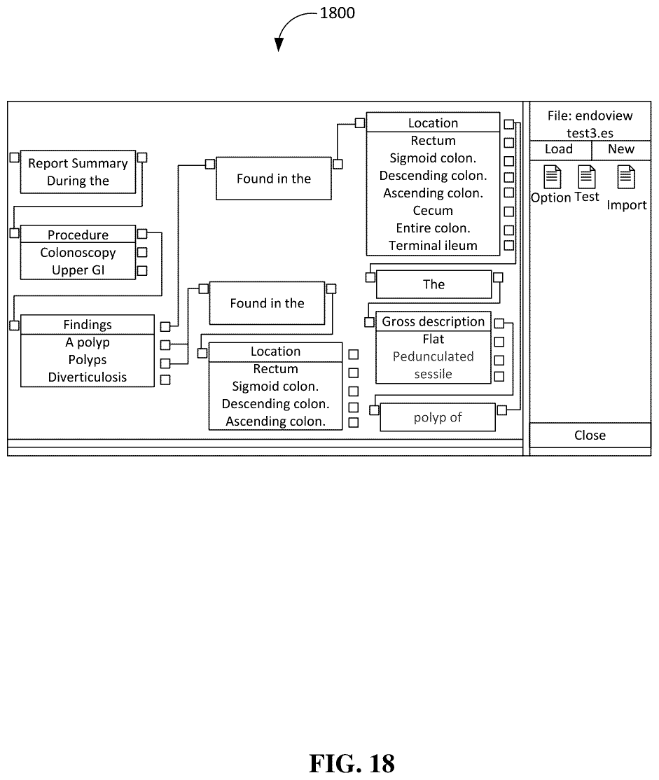

FIG. 18 is a user interface 1800 for facilitating voice-activated selections of options displayable in heads-up displays for the provisioning of the reports associated with the medical procedures, in accordance with some embodiments.

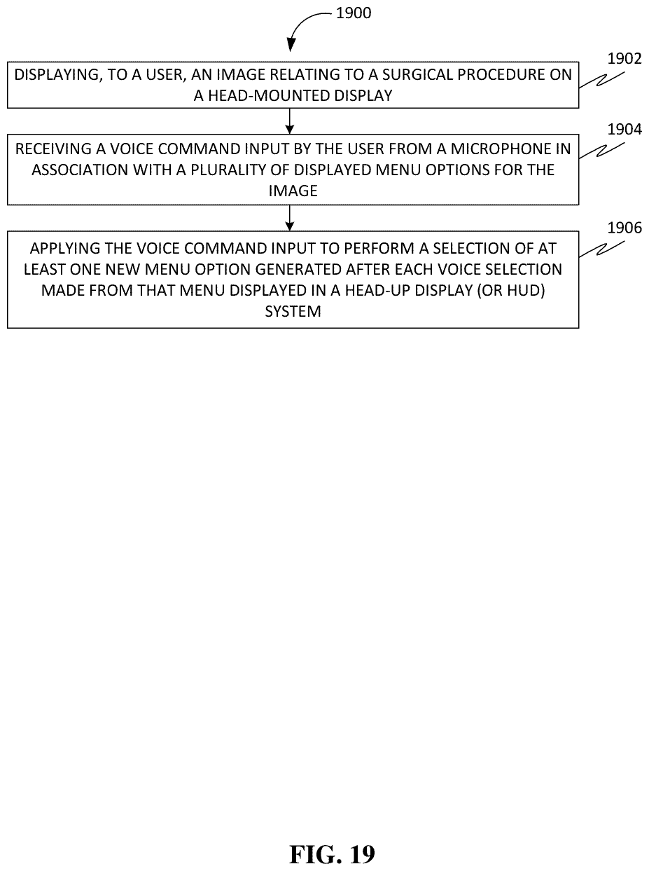

FIG. 19 is a flowchart of a method 1900 for facilitating voice-activated selections of options displayable in heads-up displays, in accordance with some embodiments.

Further, at 1902, the method 1900 may include a step of displaying, to a user, an image relating to a surgical procedure on a head-mounted display.

Further, at 1904, the method 1900 may include a step of receiving a voice command input by the user from a microphone in association with a plurality of displayed menu options for the image.

Further, at 1906, the method 1900 may include a step of applying the voice command input to perform a selection of at least one new menu option generated after each voice selection made from that menu displayed in a head-up display (or HUD) system.

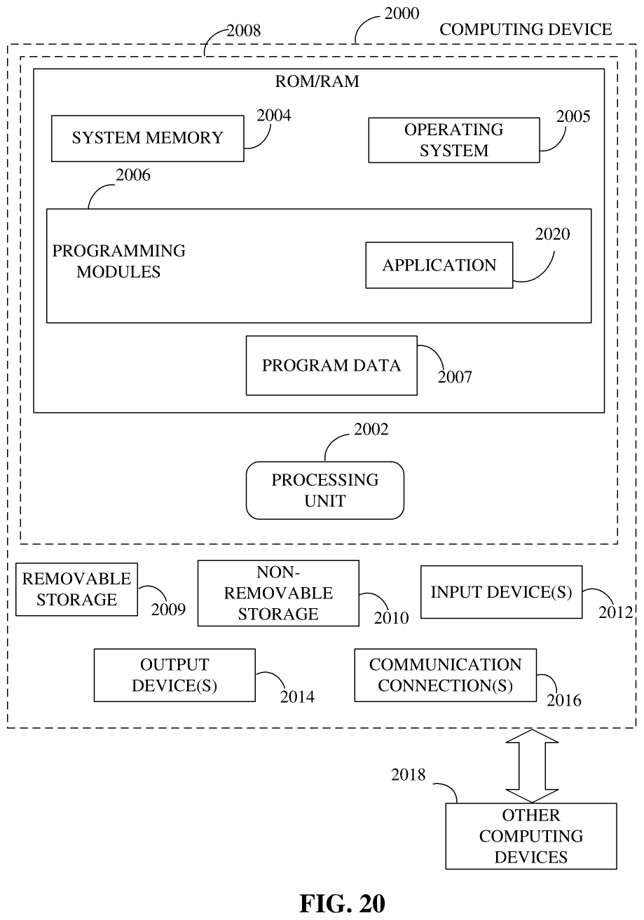

With reference to FIG. 20, a system consistent with an embodiment of the disclosure may include a computing device or cloud service, such as computing device 2000. In a basic configuration, computing device 2000 may include at least one processing unit 2002 and a system memory 2004. Depending on the configuration and type of computing device, system memory 2004 may comprise, but is not limited to, volatile (e.g. random-access memory (RAM)), non-volatile (e.g. read-only memory (ROM)), flash memory, or any combination.

System memory 2004 may include operating system 2005, one or more programming modules 2006, and may include a program data 2007. Operating system 2005, for example, may be suitable for controlling computing device 2000's operation. In one embodiment, programming modules 2006 may include image-processing modules, machine learning modules, sound processing modules, etc. Furthermore, embodiments of the disclosure may be practiced in conjunction with a graphics library, other operating systems, or any other application program and is not limited to any particular application or system. This basic configuration is illustrated in FIG. 20 by those components within a dashed line 2008.

Computing device 2000 may have additional features or functionality. For example, computing device 2000 may also include additional data storage devices (removable and/or non-removable) such as, for example, magnetic disks, optical disks, or tape. Such additional storage is illustrated in FIG. 20 by a removable storage 2009 and a non-removable storage 2010. Computer storage media may include volatile and non-volatile, removable, and non-removable media implemented in any method or technology for storage of information, such as computer-readable instructions, data structures, program modules, or other data. System memory 2004, removable storage 2009, and non-removable storage 2010 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD), other optical storage, magnetic cassettes, magnetic tape, magnetic disk storage or other magnetic storage devices, or any other medium which can be used to store information and which can be accessed by computing device 2000. Any such computer storage media may be part of device 2000. Computing device 2000 may also have input device(s) 2012 such as a keyboard, a mouse, a pen, a sound input device, a touch input device, a location sensor, a camera, a biometric sensor, etc. Output device(s) 2014 such as a display, speakers, a printer, etc. may also be included. The aforementioned devices are examples and others may be used.

Computing device 2000 may also contain a communication connection 2016 that may allow device 2000 to communicate with other computing devices 2018, such as over a network in a distributed computing environment, for example, an intranet or the Internet. Communication connection 2016 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 2004, including operating system 2005. While executing on processing unit 2002, programming modules 2006 (e.g., application 2020 such as a media player) may perform processes including, for example, one or more stages of methods, algorithms, systems, applications, servers, databases as described above. The aforementioned process is an example, and processing unit 2002 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

What is claimed is:

1. A system for provisioning reports associated with medical procedures, the system comprising:
at least one device comprising at least one output device and at least one input device, wherein the at least one output device is configured for presenting at least one medical data associated with at least one medical procedure, wherein the at least one input device is configured for detecting at least one input associated with the at least one medical procedure from at least one user;
a processing device communicatively coupled with the at least one device, wherein the processing device is configured for:
generating at least one command from the at least one user based on the detecting;
analyzing the at least one command;
selecting at least one portion of the at least one medical data based on the analyzing of the at least one command;
determining at least one operation required to be performed for the provisioning of the reports for the at least one medical procedure based on the selecting of the at least one portion of the at least one medical data and the analyzing of the at least one command;
performing the at least one operation based on the determining of the at least one operation; and
generating at least one report for the at least one medical procedure using at least one template based on the performing of the at least one operation; and
a storage device communicatively coupled with the processing device, wherein the storage device is configured for storing the at least one report.

2. The system of claim 1, wherein the processing device is further configured for:
generating a menu structure comprising at least one hierarchy level, wherein each of the at least one hierarchy level comprises a plurality of menu options corresponding to each of the at least one hierarchy level; and
selecting a menu option from the plurality of menu options for each of the at least one hierarchy level based on the at least one command, wherein the determining of the at least one operation is further based on the selecting of the menu option for each of the at least one hierarchy level, wherein the at least one output device is further configured for presenting the menu structure based on the generating of the menu structure.

3. The system of claim 2, wherein the processing device is further configured for analyzing the at least one medical data, wherein the generating of the menu structure is further based on the analyzing of the at least one medical data.

4. The system of claim 2, wherein the at least one hierarchy level comprises a first hierarchy level, wherein the plurality of menu options comprises a plurality of first menu options for the first hierarchy level, wherein the presenting of the menu structure further comprises presenting the menu structure with the first hierarchy level, wherein the selecting of the menu option from the plurality menu options for each of the at least one hierarchy level comprises:
selecting a first menu option from the plurality of menu options for the first hierarchy level based on a first command of the at least one command; and
selecting a subsequent menu option from a plurality of subsequent menu options for each of at least one subsequent hierarchy level of the at least one hierarchy level based on at least one subsequent command of the at least one command, wherein the generating of the menu structure further comprises generating the at least one subsequent hierarchy level after the first hierarchy level based on the selecting of the first menu option from the plurality of first menu options for the first hierarchy level, wherein each of the at least one subsequent hierarchy level comprises the plurality of subsequent menu options for each of the at least one subsequent hierarchy level, wherein the presenting of the menu structure further comprises presenting the menu structure with the at least one subsequent hierarchy level based on the generating of the at least one subsequent hierarchy level.

5. The system of claim 2, wherein the at least one operation comprises a generation of the at least one description for the at least one portion of the at least one medical data based on the menu option selected from the plurality of menu options for each of the at least one hierarchy level, wherein the at least one description corresponds to the menu option selected for each of the at least one hierarchy level.

6. The system of claim 2, wherein the processing device is further configured for:
determining at least one template element based on the selecting of the menu option for each of the at least one hierarchy level, wherein the at least one template element corresponds to the menu option for each of the at least one hierarchy level; and
generating the at least one template based on the determining of the at least one template, wherein the generating of the at least one report is further based on the generating of the at least one template.

7. The system of claim 1, wherein the at least one device comprises at least one of a head mountable device, an ophthalmic device, and an implantable device.

8. The system of claim 1, wherein the at least one input device comprises at least one microphone, wherein the at least one input comprises at least one utterance of the at least one user.

9. The system of claim 8, wherein the at least one input device further comprises at least one sensor, wherein the at least one sensor is configured for detecting at least one action performed by the at least one user during the at least one medical procedure, wherein the processing device is further configured for:
generating at least one action data for the at least one action based on the detecting of the at least one action; and
analyzing the at least one action data, wherein the performing of the at least one operation is further based on the analyzing of the at least one action data.

10. The system of claim 1, wherein the processing device is further configured for generating at least one content based on the at least one portion of the at least one medical data and the at least one command, wherein the generating of the at least one report is further based on the at least one content.

11. A method for provisioning reports associated with medical procedures, the method comprising:
presenting, using at least one output device of at least one device, at least one medical data associated with at least one medical procedure;
detecting, using at least one input device of the at least one device, at least one input associated with the at least one medical procedure from at least one user;
generating, using a processing device, at least one command from the at least one user based on the detecting;
analyzing, using the processing device, the at least one command;
selecting, using the processing device, at least one portion of the at least one medical data based on the analyzing of the at least one command;
determining, using the processing device, at least one operation required to be performed for the provisioning of the reports for the at least one medical procedure based on the selecting of the at least one portion of the at least one medical data and the analyzing of the at least one command;
performing, using the processing device, the at least one operation based on the determining of the at least one operation;
generating, using the processing device, at least one report for the at least one medical procedure using at least one template based on the performing of the at least one operation; and
storing, using a storage device, the at least one report.

12. The method of claim 11 further comprising:
generating, using the processing device, a menu structure comprising at least one hierarchy level, wherein each of the at least one hierarchy level comprises a plurality of menu options corresponding to each of the at least one hierarchy level; and
selecting, using the processing device, a menu option from the plurality of menu options for each of the at least one hierarchy level based on the at least one command, wherein the determining of the at least one operation is further based on the selecting of the menu option for each of the at least one hierarchy level, wherein the at least one output device is further configured for presenting the menu structure based on the generating of the menu structure.

13. The method of claim 12 further comprising analyzing, using the processing device, the at least one medical data, wherein the generating of the menu structure is further based on the analyzing of the at least one medical data.

14. The method of claim 12, wherein the at least one hierarchy level comprises a first hierarchy level, wherein the plurality of menu options comprises a plurality of first menu options for the first hierarchy level, wherein the presenting of the menu structure further comprises presenting the menu structure with the first hierarchy level, wherein the selecting of the menu option from the plurality menu options for each of the at least one hierarchy level comprises:
selecting a first menu option from the plurality of menu options for the first hierarchy level based on a first command of the at least one command; and
selecting a subsequent menu option from a plurality of subsequent menu options for each of at least one subsequent hierarchy level of the at least one hierarchy level based on at least one subsequent command of the at least one command, wherein the generating of the menu structure further comprises generating the at least one subsequent hierarchy level after the first hierarchy level based on the selecting of the first menu option from the plurality of first menu options for the first hierarchy level, wherein each of the at least one subsequent hierarchy level comprises the plurality of subsequent menu options for each of the at least one subsequent hierarchy level, wherein the presenting of the menu structure further comprises presenting the menu structure with the at least one subsequent hierarchy level based on the generating of the at least one subsequent hierarchy level.

15. The method of claim 12, wherein the at least one operation comprises a generation of the at least one description for the at least one portion of the at least one medical data based on the menu option selected from the plurality of menu options for each of the at least one hierarchy level, wherein the at least one description corresponds to the menu option selected for each of the at least one hierarchy level.

16. The method of claim 12 further comprising:
determining, using the processing device, at least one template element based on the selecting of the menu option for each of the at least one hierarchy level, wherein the at least one template element corresponds to the menu option for each of the at least one hierarchy level; and
generating, using the processing device, the at least one template based on the determining of the at least one template, wherein the generating of the at least one report is further based on the generating of the at least one template.

17. The method of claim 11, wherein the at least one device comprises at least one of a head mountable device, an ophthalmic device, and an implantable device.

18. The method of claim 11, wherein the at least one input device comprises at least one microphone, wherein the at least one input comprises at least one utterance of the at least one user.

19. The method of claim 18 further comprising:
detecting, using at least one sensor of the at least one input device, at least one action performed by the at least one user during the at least one medical procedure;
generating, using the processing device, at least one action data for the at least one action based on the detecting of the at least one action; and
analyzing, using the processing device, the at least one action data, wherein the performing of the at least one operation is further based on the analyzing of the at least one action data.

20. The method of claim 11 further comprising generating, using the processing device, at least one content based on the at least one portion of the at least one medical data and the at least one command, wherein the generating of the at least one report is further based on the at least one content.
